(19) United States
(12) Patent Application Publication
(10) Pub. No.: US 2022/0001091 A1
(43) Pub. Date: Jan. 6, 2022

(54) System for Liquid Component Fractionation and Application Method Thereof

(71) Applicant: Prim Sigma Technologies, Inc., Beverly Hills, CA (US)

(72) Inventors: Arthur Y. Yu, Beverly Hills, CA (US); Jim J. Ye, Beverly Hills, CA (US); David E. Yu, Beverly Hills, CA (US)

(21) Appl. No.: 16/920,493

(22) Filed: Jul. 3, 2020

(51) Int. Cl. A61M 1/36; A61M 39/22; B04B 5/04; B04B 11/04

(52) U.S. Cl. CPC A61M 1/3693; A61M 39/22; B04B 5/0414; B04B 2011/046; A61M 2039/229; A61M 2202/0427; A61M 2202/0415; B04B 11/04

(57) ABSTRACT

A system for liquid component fractionation includes a first container, a second container, a tunnel connecting member and a stopcock valve. The stopcock valve is a three-way valve disposed at the tunnel connecting member and is rotatable to align one of three ports of the stopcock valve to a collection outlet member of the tunnel connecting member, so as to facilitate collection of a fractionated layer from a liquid after the system is centrifuged.

### NOTICE OF COPYRIGHT

A portion of the disclosure of this patent document contains materials that are subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to isolation and purification of biological materials, and more particularly to a system introducing a stopcock valve for liquid component fractionation and application methods thereof. More specifically, the fractionation system can be employed for isolation and purification of Platelet-Rich Plasma (PRP) from whole blood, fractionation of fat components, lipids fractionation, and other fractionation situations, while there are differences in relative weights among the fractions in a liquid solution or liquid mixture.

### Description of Related Arts

PRP (Platelet-Rich Plasma) works by delivering a supra-physiologic amount of growth factors and cytokines contained within the platelet granules. The many growth factors in the platelet granules include TGF-β1, PDGF, β-FGF, VEGF, EGF, IGF-1, etc., which explains why PRP could be used for many clinical indications (De Pascale M R, Sommese L, Casamassimi A, Napoli C. Platelet derivatives in regenerative medicine: an update. Transfus Med Rev. 2015;29:52-61). In orthopedic medicine, PRP injection therapy is promising with clear evidence of efficacy and safety, based on the fact that PRP injections are being used to treat torn tendons, tendinitis, muscle injuries, arthritis, and joint injuries. For chronic wounds, PRP concentrates applied on the wound surface could provide accelerated healing. PRP injection is also being worked on for injecting into the scalp for hair regrowth, and encouraging data are being presented in many publications (Gentile P, Garcovich S, Bielli A, Scioli M G, Orlandi A, and Cervelli V. The Effect of Platelet-Rich Plasma in Hair Regrowth: A Randomized Placebo-Controlled Trial Stem Cells. Transl Med. 2015 November; 4(11): 1317-1323). Overall, the usefulness of the various growth factors contained in PRP makes it a very useful tool in the field of orthopedics, general surgery, plastic surgery, aesthetic medicine and dermatology, and etc.

The normal human platelet count ranges anywhere from 120,000 to 450,000/μL of blood. Because platelets exist in an inactive, suspension form in the blood, the isolation of them from the other components of the blood is preferably carried out by physical means, such as centrifugation, in order to avoid accidentally activating platelets prematurely.

In general, platelets could be isolated by two different approaches after the blood is mixed with an anticoagulant:

(1). Centrifugation at low g force, such as 100 g (“soft spin”). At these settings, the platelets remain suspended in the plasma. The supernatant is concentrated two folds in platelet numbers because red blood cells (RBC) account for about 45% of the total volume. This PRP supernatant is removed from the condensed red cells, then centrifuged at a higher g force (hard spin) to pellet the platelets from the plasma.

(2). Centrifugation at a high g force (“hard spin”), such as 500 g and above, to begin with. This causes the platelets to become separated from the plasma. Because platelets are lighter than leukocytes and red cells, with red cells being the heaviest, they are suspended in the middle-placed “buffy coat” layer, which includes the platelets and the white blood cells, as show in FIG. 1 of the drawings. The “buffy coat” is collected, transferred into a new test tube, suspended in plasma with a small amount of contaminating RBC and, then centrifuged again with a “soft spin” to separate the platelets and plasma from the red and white blood cells (New trends in the preparation and storage of platelets, Hogman C F, Transfusion, 1992 January; 32(1):3-6).

PRP preparations are typically further categorized into leukocyte-rich PRP (LR-PRP) preparations, defined as having a neutrophil concentration above baseline, and leukocyte-poor PRP (LP-PRP) preparations, characterized by having a leukocyte (neutrophil) concentration below baseline (Le A D K, Enweze L, DeBaun M R, and Dragoo J L. Current Clinical Recommendations for Use of Platelet-Rich Plasma. Curr Rev Musculoskelet Med. 2018 December; 11(4): 624-634). They are found to have different clinical indications for orthopedic medicine.

There is no consensus on the optimal PRP preparation with respect to the concentrations of specific blood components. Currently, many different commercial PRP harvesting systems are available on the market. As such, variation exists in the PRP collection protocols and preparation characteristics depending on the individual commercial system, giving each PRP system unique properties. The commercial systems often differ in their platelet capture efficiency, isolation method (one- or two-step centrifugation), the speed of centrifugation, and the type of collection tube system and the operation (Le A D K, Enweze L, DeBaun M R, and Dragoo J L. Current Clinical Recommendations for Use of Platelet-Rich Plasma. Curr Rev Musculoskelet Med. 2018 December; 11(4): 624-634) (Degen R M, Bernard J A, Oliver K S, Dines J S. Commercial separation systems designed for preparation of platelet-rich plasma yield differences in cellular composition. HSS J. 2017;13:75-80) (Magalon J, Bausset O, Serratrice N, Giraudo L, Aboudou H, Veran J, Magalon G, Dignat-Georges F, Sabatier F. Characterization and comparison of 5 platelet-rich plasma preparations in a single-donor model. Arthroscopy. 2014; 30:629-638). Generally, fresh whole blood is drawn and mixed with an anticoagulant. Next, centrifugation(s) separates red blood cells (RBCs) from platelet-poor plasma (PPP) and the “buffy coat,” which contains concentrated platelets and leukocytes, as shown in FIG. 1 of the drawings. The platelets are isolated using various conventional methods. The obtained PRP product can then either be directly injected into the patient or be activated by adding either calcium or thrombin, causing the platelets to degranulate and release the growth factors (Marx R E. Platelet-rich plasma (PRP): what is PRP and what is not PR? Implant Dent. 2001; 10:225-228).

However, in addition to some patient-specific factors such as people to people variations, health states or medications taken, different commercial systems influence both the quality and quantity of the harvested PRP, creating challenges in the downstream applications (Le A D K, Enweze L, DeBaun M R, and Dragoo J L. Current Clinical Recommendations for Use of Platelet-Rich Plasma. Curr Rev Musculoskelet Med. 2018 December; 11(4): 624-634). Some of the main issues with the current available commercial kits include one or more of the following: long preparation time, cumbersome to use, low harvest rate, too much contamination by red blood cells, lack of ability to concentrate platelets beyond two folds, lack of ability to differentially isolate LR-PRP and LP-PRP, disturbance of the “buffy coat” during the collection stage, inconsistent results, and not to mention that many require the purchase of a separate special centrifuge, other than a regular, easily available benchtop centrifuge.

Meanwhile, in the field of fat fractionation, the main issue would be to increase the efficiency of separating oil from the fat component. The fat could be a preparation of regular fat, nano fat, or stromal vascular fraction (SVF) which harbors the fat stem cells.

Typically, surgeons would use either the conventional sedimentation method or a simple centrifugation method for fat graft purification. However, the sedimentation method, letting the lipoaspirate sit for 10 to 30 minutes, does not allow removal of oil, and the amount of water and RBC sedimented to the bottom is not consistent. Not to mention that in some fat harvests, the oil content could be anywhere from 5 to 80%, making the results of fat grafting unpredictable. Because of this and the fact that free lipids might harm the survival of fat grafts, it is better to use centrifugation to fractionate the fat allowing oil to be removed. However, what troubles the surgeons about the centrifugation method is the limited ability of removing the extremely sticky oil from the top of a tube. Even though there are quite a few commercial systems available for removing oil, most of these are either too expensive, or hard to use. And some do not even work as claimed.

As far as nano fat is concerned, the situation is even more complicated. Nano fat is created by first shuffling the harvested fat 30 times through a small “holed” (1.2 mm) Tulip LuerTransfer® (Tulip Medical Products, San Diego, Calif.) and then, subjecting the resulting fat to be pressed through a 400 micron screen. The recommendation is to use the emulsified product directly, or as is. However, this practice may not be sound considering what the components are after a centrifugation process, as shown in FIG. 2. Almost 30 to 50% of the fat will be destroyed by the shuffling and screening processes, releasing free lipids (oil). It is found that this oil is even harder to remove than the oil fractionated in the preparation of regular fat grafts, probably because emulsification takes place before the centrifugation. However, ideally, this oil byproduct should be isolated out before a quality fat grafting is performed. Because this nano fat preparation is so fine in sizes, many of the commercial systems cannot even be used, as a result of significant loss of the sample during the cleaning process.

Autologous fat stromal vascular fraction (SVF) contains most of the stromal stem cells. It could be prepared mechanically by ultrasonic sonication, homogenization, fine trituration, mortar and pestle treatment, etc. Traditionally, it has always been a difficult task to harvest SVF in a clean way. While the number of SVF cells are in the numbers of 10⁵ to 10⁶ per ml of fat, the single cell numbers in the finely homogenized fractions in suspension are in the ranks of 10⁹. Even a diminutive, almost ignorable amount of contamination of the SVF by the above-mentioned fractions would drastically undermine the validity of the SVF cell counts at the end of the purification, and the subsequent treatment regimens or related experiments. This kind of contamination takes place very easily and very frequently with most, if not all, of the systems currently in use, for reasons such as the contaminating fractions sticking to the walls of the tube/bottle, or some almost invisible small pieces breaking away from the main fraction layer during the SVF collection. These are further complicated by the fact that the morphology of pre-adipocytes and small fat cells are not much different from the SVF cells when in suspension. Considering these, it is no wonder that the numbers of harvested “stem cell” from fat vary from 10⁴ to 2.2×10⁷ (WO2015005871A1, WO2014000031A1, and U.S. Pat. No. 8,440,440B2) among different publications. And this fact significantly hampers the progress in the field of fat stem cell research and clinical applications. As far as we know, currently, there is no reliable method or commercially available system for the purpose of purifying SVF without any risk of contamination by the other fractions.

Autologous fat is harvested routinely, i.e., with suction-assisted lipectomy or liposuction. Since the water content in a fat preparation varies depending on the amount of tumescent fluid used, the amount of time waited, and the inter-personal differences, most surgeons prefer to centrifuge down the fat to remove the free oil and water content, in order to perform fat grafting or other procedures in a more predictable way. The centrifugation force could be anywhere from a few g to 10,000 g, although at higher g forces, the survival of the fat grafts may not be as good (Strong A L, Cederna P S, Rubin J P, Coleman S R, and Levi B. The Current State of Fat Grafting: A Review of Harvesting, Processing, and Injection Techniques. Plast Reconstr Surg. 2015 October; 136(4): 897-912). The centrifugation could be 5 to 10 minutes. The usual speed to be used is around 500 g.

However, it is not an easy task to separate out the desired fat fraction after the fat is centrifuged, as shown in FIG. 2 of the drawings. While most of the water content in the infranatant could be removed, a lot of the contaminating RBC could be mixed into the final fat preparation. More importantly, the supernatant oil fraction is very sticky and is hard to be poured off or suctioned out (Simonacci F, Bertozzi N, PioGrieco M, Eugenio Grignaffini E, and Raposio E. Procedure, applications, and outcomes of autologous fat grafting. Annals of Med and Surg. 2017; 8:49-60). Blotting with cotton pieces does not work either.

Quite a few commercial systems (Salinas H M, Fernandes J R, Westman A M, Colwell A S, Broelsch G F, McCormack M C, Randolph M A, and Austen W G. Comparative analysis of processing methods in fat grafting. Plast Recon Surg., 2014 June; 134(4):1097) are available, but most do not provide satisfactory results in terms of separating oil from fat. Even if one could visually confirm that separation of oil from fat is already achieved after centrifugation, the mixing of oil and fat would still take place, likely due to the extremely high affinity of oil to fat cells. Furthermore, many of the commercial systems are messy and cumbersome to use. Since studies showed that contaminating free lipids and RBC could be harmful to the grafted fat, it would be advantageous if one could use a device to fractionate the various components of the fat preparations in a clean and easy way.

### SUMMARY OF THE PRESENT INVENTION

The present invention is advantageous in that it provides a system for liquid component fractionation and application method thereof, wherein by operation of a stopcock valve, it is easy and effective to fractionate components of a liquid solution or liquid mixture.

Another advantage of the present invention is that it provides a system for liquid component fractionation and application method thereof, wherein the system functions as a centrifugation tube that allows liquid state components being fractionated, with subsequent sequestration of desired one or more fractions being collected from a collection port, while various components in the liquid have different relative weights.

Another advantage of the present invention is that it provides a system for liquid component fractionation and application method thereof, wherein the system is suitable for the isolation and purification of PRP from whole blood, so that the purified PRP could be subsequently used for various clinical and experimental purposes.

Another advantage of the present invention is that it provides a system for liquid component fractionation and application method thereof, wherein the system for PRP isolation from the blood is easy to use, saves time, increases yields, provides the capability of concentrating PRP up to 15 to 20 folds or even more, facilitates the differential isolation of LR-PRP and LP-PRP, improves the consistency of the PRP production, and avoids the need for additional special equipment purchase because the system of the present invention could easily fit in most standard tabletop centrifuges.

Another advantage of a system for liquid component fractionation and application method thereof of the present invention is that, the system is suitable for the fractionation of fat components, including, but not limited to, removal of contaminants from fat, refinement of nano fat, and purification of fat stromal vascular fraction (SVF).

Another advantage of a system for liquid component fractionation and application method thereof of the present invention is that, due to the fact that the present invention is very versatile in nature, the system could also be used in the preparation of other materials or substances in other fields and industries, such as lipids fractionation, so long as there are differences in relative weights among the fractions in the liquid solution or liquid mixture.

Additional advantages and features of the invention will become apparent from the descriptions that follow, and may be realized by means of the instrumentation and the various combinations of modifications particularly pointed out in the appended claims.

According to the present invention, the foregoing and other objects and advantages are attained by a system for liquid component fractionation, comprising:

a first container having a first containing cavity;

a second container having a second containing cavity; and

a tunnel connecting member, provided between the first container and the second container, comprising at least one tunnel body having one or more connecting tunnels communicating with the first container and the second container, and a collection outlet member, having a collection port, coupled to the at least one tunnel body, and

a stopcock valve, provided at the middle of the tunnel connecting member, having three ports configured for selectively aligning with the one or more connecting tunnels and the collection port.

According to another aspect of the present invention, the present invention further provides a method for liquid component fractionation through a system which comprises a first container, a second container, a tunnel connecting member provided between the first container and the second container, and a stopcock valve provided at the middle of the tunnel connecting member, wherein the method comprises the following steps.

(a) Centrifuge the system which is filled with a liquid to separate the liquid into a plurality of fractionated layers.

(b) Operate the stopcock valve to allow at least one of the plurality of fractionated layers to be collected through a collection outlet member of the tunnel connecting member.

Still further objects and advantages will become apparent from a consideration of the ensuing descriptions and drawings.

These and other objectives, features, and advantages of the present invention will become apparent from the following detailed descriptions, the accompanying drawings, and the appended claims.

### BRIEF DESCRIPTION OF THE DRAWINGS

FIG. 1 is a perspective view of a conventional test tube which is centrifuged for isolating PRP.

FIG. 2 is a perspective view of a conventional test tube which is centrifuged for separating out a desired fat fraction.

FIG. 3 is a perspective view of a system for liquid component fractionation according to a preferred embodiment of the present invention.

FIG. 4, viewed from an angle that is turned 90 degrees clockwise from the front view of FIG. 3, is another enlarged perspective view of a stopcock valve and a tunnel connecting member of the system for liquid component fractionation according to the above preferred embodiment of the present invention.

FIG. 5 is an enlarged front perspective view of the stopcock valve and the tunnel connecting member of the system for liquid component fractionation according to the above preferred embodiment of the present invention.

FIG. 6A is an enlarged side view of a valve plug of the system for liquid component fractionation according to the above preferred embodiment of the present invention.

FIG. 6B is an enlarged side view of the valve plug (from the opposite side of FIG. 6A) of the system for liquid component fractionation according to the above preferred embodiment of the present invention.

FIG. 7 is a perspective view illustrating three fractionation phases in the system for liquid component fractionation, when the system is filled with blood after centrifugation according to the above preferred embodiment of the present invention.

FIG. 8 is a perspective view illustrating the utilization of a plunger shaft for pushing a buffy coat towards the stopcock valve of the system for liquid component fractionation according to the above preferred embodiment of the present invention.

FIG. 9, viewed from an angle that is turned 90 degrees clockwise from the front view of FIG. 8, is another enlarged perspective view illustrating the collection of the PRP by a syringe which is coupled to a collection outlet member of the system for liquid component fractionation according to the above preferred embodiment of the present invention. Also shown is the differential positioning of the LP-PRP and LR-PRP.

FIG. 10 is a perspective view illustrating the collection of PRP by a syringe which is coupled to the collection outlet member of the system for liquid component fractionation according to the above preferred embodiment of the present invention.

FIG. 11, viewed from an angle that is turned 90 degrees clockwise from the front view of FIG. 8, is another enlarged perspective view illustrating the collection of the PRP by a syringe which is coupled to a collection outlet member of the system for liquid component fractionation according to the above preferred embodiment of the present invention, wherein the differential positioning of the LP-PRP and LR-PRP is shown.

FIG. 12 illustrates a Table showing the comparison of PRP isolation by the system for liquid component fractionation according to the above preferred embodiment of the present invention with some commercial systems in terms of volume requirement, time spent, concentration capability and capture efficiency.

FIGS. 13, 14 and 15 are perspective views illustrating a procedure for fat graft preparation from lipoaspirates by the system for liquid component fractionation according to the above preferred embodiment of the present invention.

FIG. 16 is a perspective view illustrating a procedure for nano fat processing by the system for liquid component fractionation according to the above preferred embodiment of the present invention.

FIGS. 17A and 17B are perspective views illustrating a procedure for Autologous fat stromal vascular fraction (SVF) harvesting by the system for liquid component fractionation according to the above preferred embodiment of the present invention.

FIG. 18 is a perspective view illustrating the system for liquid component fractionation according to a first alternative mode of the above preferred embodiment of the present invention.

FIGS. 19A and 19B are microscopic views of the cell culture of the first generation of the fat SVF harvested using the system for liquid component fractionation according to the first alternative mode of the above preferred embodiment of the present invention.

FIG. 20A is a perspective view illustrating the system for liquid component fractionation according to the second alternative mode of the above preferred embodiment of the present invention.

FIG. 20B is a perspective view illustrating the system for liquid component fractionation according to the third alternative mode of the above preferred embodiment of the present invention.

FIG. 20C is a perspective view illustrating the system for liquid component fractionation according to the fourth alternative mode of the above preferred embodiment of the present invention.

FIG. 21A is an enlarged schematic view illustrating a stopcock valve of the system according to a fifth alternative mode of the above preferred embodiment of the present invention.

FIG. 21B is an enlarged schematic view illustrating a valve plug of the 3-way stopcock valve provided in the tunnel connecting member of the system, viewing from a 30 degree angle upward along the “arrow” in FIG. 21A, according to the fifth alternative mode of the above preferred embodiment of the present invention.

FIG. 22A is an enlarged schematic view illustrating a stopcock valve of the system according to the fifth alternative mode of the above preferred embodiment of the present invention.

FIG. 22B is an enlarged schematic view illustrating a valve plug of the stopcock valve provided in the tunnel connecting member of the system according to the fifth alternative mode of the above preferred embodiment of the present invention.

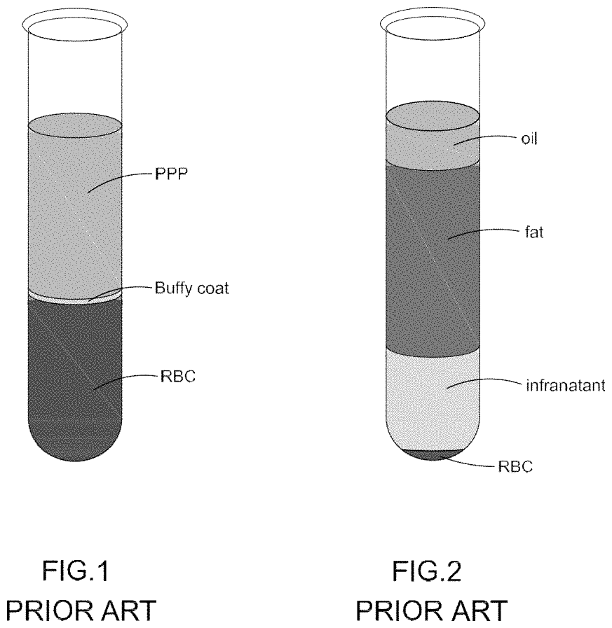

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

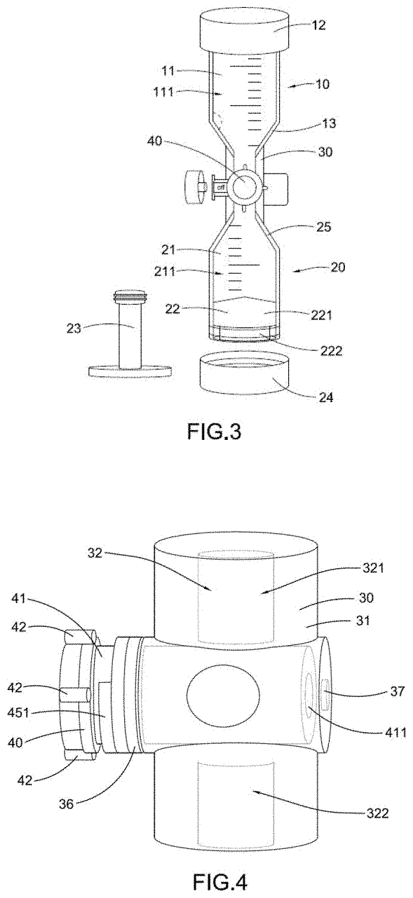

Referring to FIG. 3 to FIG. 11 of the drawings, a system for liquid component fractionation according to a preferred embodiment of the present invention is illustrated. The system comprises a first container 10, a second container 20, a tunnel connecting member 30 connecting the first container 10 and the second container 20, and a stopcock valve 40 disposed at the tunnel connecting member 30. When a liquid sample containing a plurality of fractions which have differences in relative weight is filled into the system, the system is centrifuged to produce a plurality of fractionated phases that can be subsequently separated and collected. Particularly important is that the system could be a disposable system, which could be made of polypropylene, polyethylene, polycarbonate or other alternative materials, for the isolation of specific components in a blood or tissue sample.

The first container 10 and the second container 20 can be embodied as two transparent conical tubes coupled to an “extra-large” 3-way stopcock valve 40. Mechanically and methodologically, the system can function as a centrifugation tube that allows liquid state components to be fractionated, with subsequent sequestration of one or more fractions from a collection port, while the various components in a liquid have different relative weights.

According to this preferred embodiment, the first container 10 comprises a first container body 11 with a first containing cavity 111, and a first cap 12 which can be mounted to the distal end of the first container body 11. The second container 20 comprises a second container body 21 with a second containing cavity 211, a movable member 22 (similar to the plunger tip of a conventional syringe, made of rubber) disposed in the second containing cavity 211, a plug member 23 (similar to the plunger shaft of a conventional syringe) which can be screwed onto the bottom supporting member 222 of the movable member 22 and when operating, can push the movable member 22 in the lower containing body 21, and a cap 24 mountable to the distal end of the second container body 21.

Referring to FIGS. 3 to 5, the tunnel connecting member 30 comprises a tunnel body 31 with a connecting tunnel 32 communicating with the first containing cavity 111 of the first container body 11 and the second containing cavity 211 of the second container body 21, through proximal end portions of the first and second container 10, 20 respectively, a side wing 33 extending sideward from the tunnel body 31 at one side thereof for abutting the wall of the centrifugation container for better stability of the system while being centrifuged, and a collection outlet member 34 provided at the opposite side of the tunnel body 31 defining a collecting port 341 for collecting a desire fractionated phase.

The stopcock valve 40 is provided at the tunnel connecting member 30 and comprises a valve plug 41, with preferably a first interconnecting port 401, a second interconnecting port 402 and a third interconnecting port 403 which are selectively aligned with the first containing cavity 111, the second containing cavity 211, and the collecting outlet member 34 respectively, when used for platelet purification. The stopcock valve 40, according to this preferred embodiment, is a valve system that comprises three valve port identifiers 42, a valve lever 43, and an “off” position identifier 44. Where the “off” position identifier 44 points that particular corresponding port will be blocked off for any flow. The three valve port identifiers 42 indicates the respective positions of the three inter-connecting ports 401, 402 and 403 of the valve plug 41.

According to an application of this preferred embodiment of the present invention, the system can be employed for liquid component fractionation, such as PRP isolation from the blood. The system is easy to use, saves time, delivers better yields, provides the capability of concentrating PRP up to 15 to 20 folds or even more, facilitates the differential isolation of LR-PRP and LP-PRP, improves the consistency of the PRP production, and avoids the need for additional special equipment purchase because the system of the present invention could easily fit in most standard tabletop centrifuges.

For PRP extraction, freshly drawn blood is mixed with an anticoagulant, for example ACD (acid-citrate-dextrose) buffer at room temperature. After the first cap 12, which is secured on the first container body 11 (screwed on to the distal end portion of the first container body 11), is unsecured and removed from the first container body 11, the blood is instilled into the system, passing the first container cavity 111, through the connecting tunnel 32 in the middle of the system, reaching the second container cavity 211. The connecting tunnel 32 has an inner diameter, preferably around 6 to 7 mm, smaller than the diameter of the first container cavity 111 and the diameter of the second container cavity 211. Practically, the inner diameter could vary from 3 to 15 mm, depending on the sizes of the first and second containers 10 and 20. In application, the first container 10 can be embodied as the upper container while the second container 20 can be embodied as the lower container, and the blood is filled into the system through the upper container.

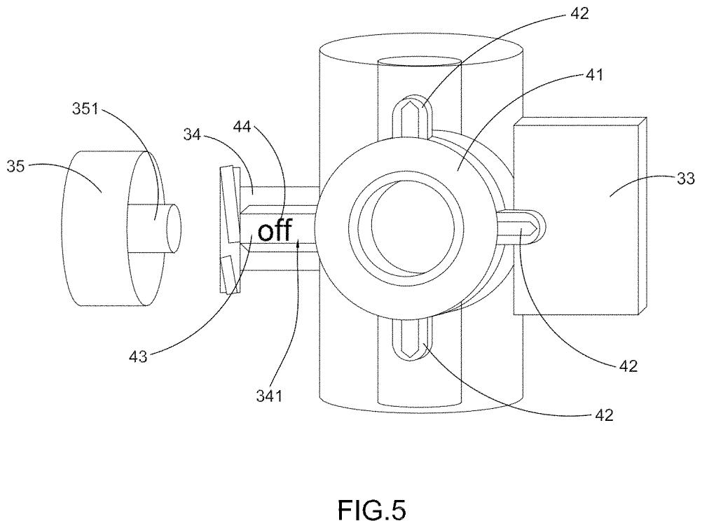

The tunnel connecting member 30 can be fortified with a thicker plastic wall (actual thickness depends on the material), for better strength. The thickened wall is provided with a side wing 33 extended from one side of the tunnel connecting member 30 and configured for the purpose of abutting the wall of the centrifugation container for better stability of the system while being centrifuged, as shown in FIG. 5.

The connecting tunnel 32 of the tunnel body 31, having a straight elongated configuration, allows the blood to flow downward from the upper first container 10 to the lower second container 20, while the valve lever 43 of the stopcock valve 40 is switched horizontally to block the collection outlet member 34. This collection outlet member 34 of the stopcock valve 40 is provided at the opposite side of connecting tunnel 31 with respect to the side wing 33. The blood then enters the second container 20 through the connecting tunnel 32 and reaching the bottom of the second container 20, which comprises the movable member 22. The movable member 22 comprises a movable member 221 similar to a regular syringe plunger rubber tip top and an engaging member 222 provided at the free end portion thereof for supporting the movable plunger tip 221. Throughout the centrifugation process, the second cap 24 is kept on to securely cover the free end portion of the second container 20, holding the engaging member 222 and providing a definitive mechanism to contain the blood content.

The cap 24 is removed after centrifugation, revealing the engaging member 222 which has inner threads, allowing a separate, detachable plug member 23 to be engaged thereto by the action of twisting-on using its outer threads at the free end portion. Once the plug member 23 is twisted on to the bottom of the member 222, they form the whole plunger complex and could now function fully, allowing precise gliding of the movable member 22 up or down the second container 20 smoothly.

The tunnel connecting member 30 further comprises a sealing cap 35 comprising a central plug 351 which fits into and provides sterility protection to the collection outlet member 34, wherein this connection can be accomplished by the Luer-lock mechanism. The position and the height of this sealing cap 35 are in such a manner that the system abuts the centrifugation container on the opposite side from the side wing 33, for a sturdy and undisturbed centrifugation. As a result, this system is very stable throughout the centrifugation process. Neither the first or the second cap 12 and 24 are air-tight, to enable the movement of liquid contents in either directions by manipulating the stopcock valve 40 only, without the need to uncap the tubes. Additionally, first and second conical slope surfaces 13 and 25 are at the proximal end portions of the first and second container 10, 20 respectively, transitioning from the first container 10 and the second container 20 to the tunnel connecting member 30 respectively, each forming a wide angle with the straight container walls at preferably 120 to 150 degrees (the workable range could be from 100 to 170 degrees), in order to facilitate the movement of the blood components in either upward or downward direction. Accordingly, no unwanted retention of the blood components will take place in the transition sections, minimizing RBC contamination at the end.

Once the blood is filled into the system of the present invention, the system is placed in a standard centrifuge container for centrifugation at 900 g for 8 minutes, at room temperature. And this combination of numbers could vary widely, as have been practiced by many. For example, the g force could be from 300 g to 1500 g, and the time could be 3 to 20 minutes. There should be no braking at the end of the centrifugation, so as not to disturb the “buffy coat”.

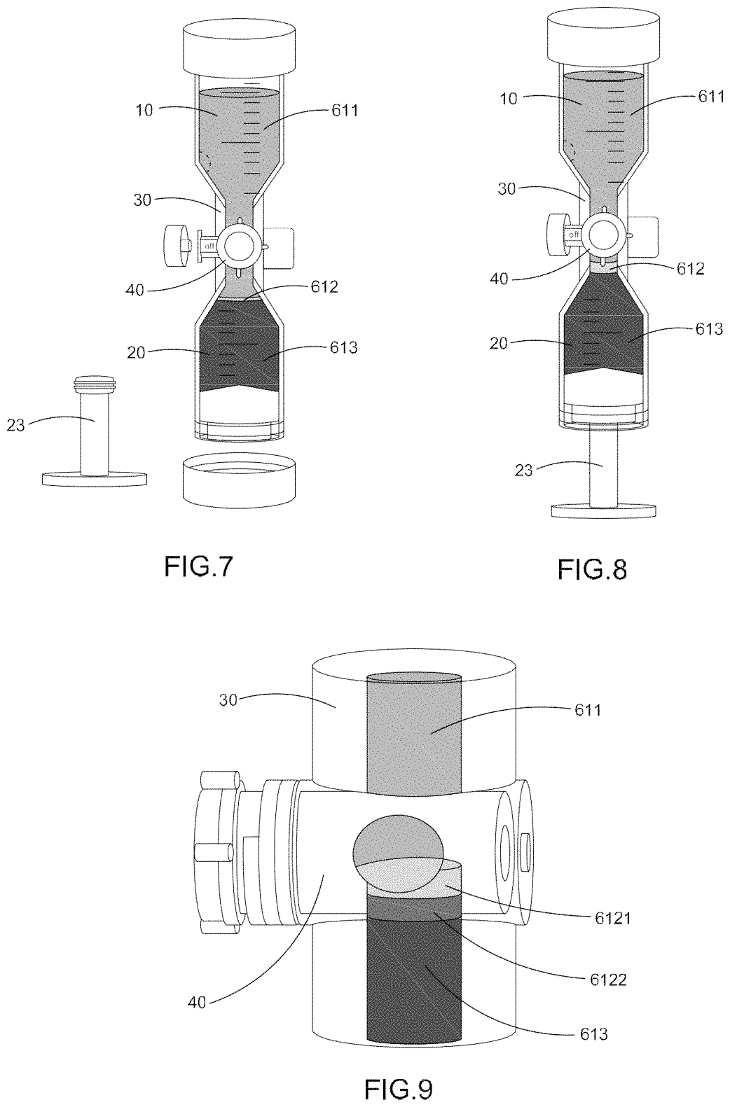

FIG. 7 shows the three fractionation phases from the centrifugation: (i) the platelet-poor-plasma (PPP) which is a first layer 611 at the top, occupying 55% of the volume; (ii) a whitish thin layer 612 of “buffy coat” in the middle section, comprising about 1% of the volume; and (iii) a third layer 613 at the bottom which are RBC condensates, containing approximately 45% of the volume.

Thereafter, referring to FIGS. 7 and 8, the bottom second cap 24 is removed and the plug member 23 is fastened onto the engaging member 222 to assemble the complete plunger complex which comprises the movable plunger tip 221, the engaging member 222 and the plug member 23. The plug member 23 is then able to be pushed gently to let the rubber head glide upward. Consequently, the “buffy coat” migrates toward the tunnel connecting member 30 until reaching the stopcock valve 40.

To isolate the platelets, the “buffy coat”, which contains platelets and leukocytes, needs to be physically separated from RBC. This is achieved by slowly pushing the plug member 23 of the plunger complex further up, pushing the “buffy coat” to pass the boarder of the first port 402 which is the bottom port of the stopcock valve 40. FIG. 9 shows the tunnel connecting member 30 with the stopcock valve 40 in place, wherein its view angle is turned clockwise 90 degrees from FIGS. 7 and 8.

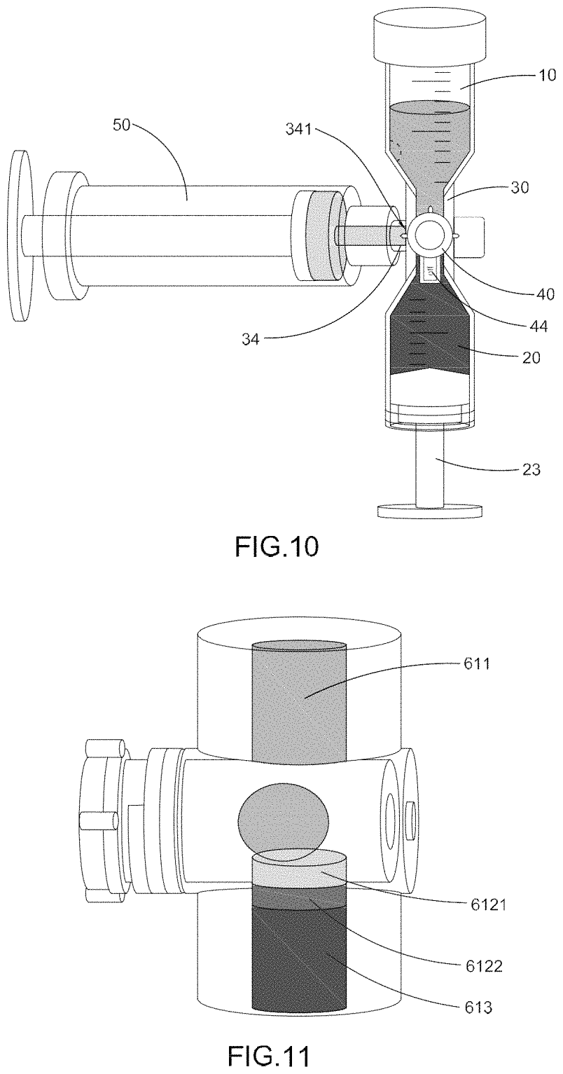

Next, further referring to FIG. 10, the system utilizes a collection syringe 50 that is connected to the collection outlet member 34. The stopcock valve 40 is then turned to block the RBC compartment in the second container 20, wherein none of the three ports 401, 402 and 403 communicates with the second container 20. After the stopcock valve 40 is shut to the RBC port, the PRP could be easily collected by withdrawing the plunger of the collection syringe 50. Note that no RBC from the second containing cavity 211 could enter the collection port/outlet or the syringe 50 once the three-way stopcock valve 40 locks down the passageway to RBC.

Since LR-PRP and LP-PRP were found to have distinctively different indications in the treatment of musculoskeletal diseases, one could easily harvest LR-PRP or LP-PRP by manipulating the position of the “buffy coat” at the threshold of the lower valve port of the stopcock valve 40, as shown in FIGS. 9 and 11. By only allowing the upper half of the “buffy coat” into the threshold of the lower vale port of the stopcock valve 40, and then turn the valve lever 43 of the valve to block out both the lower “buffy coat” and the RBC at the bottom, the layer 6121 LP-PRP is collected as shown in FIG. 11. After the collection of the LP-PRP 6121, the lever 43 of the stopcock is returned to its original position to block off the collection outlet 34. The system is subject to another round of centrifugation. This time, when the PRP is collected again from the system, it is now LR-PRP 6122, as shown in FIGS. 9 and 10.

Both FIGS. 9 and 11 are magnified views of only the tunnel connecting member 30 and the stopcock valve 40 of the invention from an angle that is turned 90 degrees clockwise from the frontal view of FIG. 8. The thick side wing 33 is purposely omitted in this figure merely for a clearer view of the fractionated components. The round opening on the valve plug 41 (inside the transparent three-way stopcock casing) facing front is one of three valve ports 401, 402, and 403 on the valve plug 41 inside the three-way-stopcock, whereas the collection port 341 as shown in FIGS. 2, 7, 8 and 10 lies on the opposite side. While this valve port is not connected to any passageway in this position with the “off” position identifier 44 aligned with the collection outlet member 34, the other two ports are connected to the first and second containing cavities 111 and 211 respectively.

To concentrate the platelets in the PRP to a higher level, the PPP could be suctioned out with minimal disturbance from the top of the first container 10 with the top first cap 12 removed. Because the stopcock valve 40 effectively closes the contaminating RBC off at the bottom, as shown in FIG. 10, more than 90% of PPP could be removed and discarded without disrupting the “buffy coat”. The removal of PPP could be done with a long needle or cannula coupled to a syringe. Accordingly, it provides at least 15 to 20 times concentration for the platelets in the final PRP. However, in case better purification or much higher concentration of platelets is desired, the harvested PRP could be subjected to a “soft spin” first to get rid of the red cells and most of the leukocytes. The resulting supernatant could then go through a round of “hard spin” to pellet down the platelets, as described earlier.

FIG. 4 shows the same structural details as in FIGS. 9 and 11. This is the side view of FIG. 3 with the device turned 90 degrees clockwise. It comprises the tunnel connecting member 30 and the stopcock valve 40 without the sample components. Both the outlet port member 34 and the valve lever 43 are not shown. The first container 10 and the second container 20 are truncated for a clearer view of the structure.

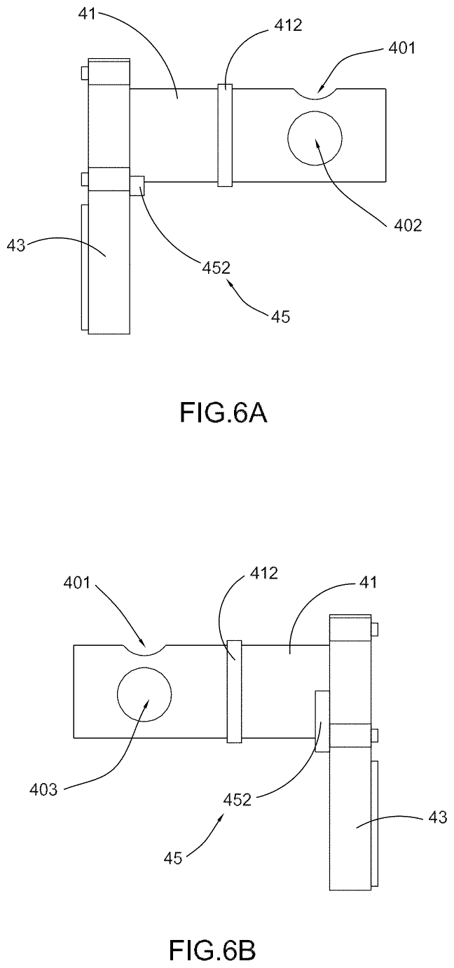

This middle section of the system of the invention comprises the stopcock valve 40 and the tunnel connecting member 30. The rotatable stopcock valve 40 is housed in the tunnel connecting member 30. The stopcock valve 40 has three interconnecting ports 401, 402 and 403. The tunnel connecting member 30 has a first passage 321 and a second passage 322, and a collection port 341. At any point of time, only two of the valve plug ports 401, 402,403 are open to each other. The stopcock valve 40 further comprises a blocking member 45 (FIG. 6A and 6B). A third connected passageway is prevented by using the blocking member 45 comprising two separate quarter-annular blocking elements, i.e. a static one 451 close to the plug handle is located on the tunnel connecting member 30, as shown in FIG. 4, and a mobile one 452 is located behind the valve lever 43 on the valve plug 41, moving along with the valve plug 41. The result of the two quarter-annular blocking elements working to block each other is that the valve lever 43 can only move 180 degrees: i.e. counterclockwise from the upper direction to the outlet, then onto the lower direction; and clockwise to turn back. Consequently, the outlet port can never be connected with both first and second passage 321 and 322 at the same time.

The valve plug 41 is essentially a cylindrical structure hugged snugly by the outside valve body. Of course, the plug handle is not encased by the tunnel connecting member 30. The tunnel connecting member 30 further comprises an annular rib 36 on the inner surface of the tunnel connecting member 30, which presses against the annular wall structure on the valve plug 41 into a sealing engagement with the wall of the tunnel connecting member 30 forming a watertight seal, as shown in FIGS. 4 and 6.

On the end portion of the tunnel connecting member 30, opposite to the collection outlet member 34, is a disk shaped and slightly raised round prominence 37 and on the end of the valve plug 41 is a slightly larger and round recession area 411, as shown in FIG. 4. At the time of fluid entrance into the plug ports, fluid entering the space between prominence 37 and the recession area 411 will press the valve plug 41 further so the watertight seal between the previously mentioned annular structures between the stopcock valve 40 and the tunnel connecting member 30 will be even tighter.

FIG. 5 shows the frontal view of the tunnel connecting member 30 and stopcock valve 40. FIGS. 6A and 6B is the left and right-side views of a bare valve plug 41, which has three interconnecting ports 401, 402 and 403, one annular ring prominence 412 and the mobile one quarter-annular blocking element 452.

Compared to various commercially available kits or methods, this invention is inexpensive, easy to use, smooth in operation, and safe to handle. It does not require a long time to obtain the final product(s), has a high yield with consistent results, and shows minimal disturbance during the harvesting stage. Because the system of the invention fits in most readily available table-top centrifuges, there is no need to buy additional expensive special centrifuge. Additionally, the contamination by RBC is minimal, and the ability to concentrate PRP up to 20 times in one spin is a powerful feature. Last but not the least, it is a significant advantage that the device could be utilized to differentially isolate LR-PRP and LP-PRP.

Depending on the requirements, the volume handled could be anywhere from 5 ml to 100+ ml. Of course, volumes at the more extreme ends need to be fitted into specially designed tubes.

The g force required for centrifugation is mostly a personal preference, so long as the numbers are not too much off. Note that one of the more important features to pay attention to is the angle in FIG. 3. Steeper conical slope surfaces 13 and 25 adjacent the proximal end portions of the first and second containers 10 and 20 simply prevent the unnecessary lodging of contaminating RBC.

The basic structure, such as the first and second container which are embodied as tubes in this preferred embodiment, could also be further modified. As long as the basic principles are observed, they could be made detachable to further facilitate the separation of samples requiring special treatments.

In summary, the present invention is simple to use and inexpensive to manufacture. The system of the present invention is adapted to be a disposable device after a single use, to avoid cross contamination. Sterilization post manufacturing is achieved by gamma-radiation or similar measures. Despite the mentioning of only PRP in the above text, it is by no means limited to these uses. In addition to PRP isolation, various fat preparations could be fractionated. In fact, anything that present differential relative weight qualities in a sample material could be separately isolated using this invention.

### SAMPLE PREPARATION EXAMPLE 1

As an Example of Blood fractionation for PRP preparation, a phlebotomist may draw blood into a syringe containing 1/10 volume of ACD or CPD buffer. The volume of blood needed depends on the usage. For most treatments, 20 ml of blood is usually sufficient. The blood is gently filled into the system from the top, with the valve lever 43 of the stopcock valve 40 blocking the collection port 341. Once the blood is filled into the tube complex of the system, the system is placed into a standard centrifuge container for centrifugation at 900 g for 8 minutes at room temperature without braking. Then, the system is removed from the centrifuge after it comes to a complete stop. FIG. 7 shows the three fractionation phases in the tube system: (i) the platelet-poor-plasma (PPP) on the top, (ii) the whitish thin layer of “buffy coat” in the middle section, and (iii) the bottom RBC condensates.

Then, at this point, the second cap 24 is removed and the plug member 23, comprising a plunger shaft, is fastened to the engaging member 222 to assemble the complete plunger complex which comprises the movable plunger tip 221, the engaging member 222 and the plug member 23. The plug member 23 is then pushed gently to let the rubber head glide upward smoothly. Consequently, the “buffy coat” migrates toward the tunnel connecting member 30, reaching the stopcock valve 40, as shown in FIG. 8.

To isolate the platelets, the “buffy coat” which contains platelets and leukocytes which need to be physically separated from RBC. This is achieved by slowly pushing the plug member 23 further up, pushing the “buffy coat” to pass the boarder of the lower valve port of the stopcock valve, as shown in FIG. 9.

Next, the collection syringe 50 is connected to the collection outlet member 34. The valve port of the stopcock valve 40 is then turned to block the RBC compartment in the second containing cavity 211. This is a very easy-to-perform motion, and no disturbance to the “buffy coat” should take place. After the valve is shut to the RBC port, the PRP could be easily collected by withdrawing the plunger of the collection syringe 50. Note that no RBC from the lower tube compartment could enter the collection port or the syringe once the three-way stopcock valve 40 locks down the passageway to RBC.

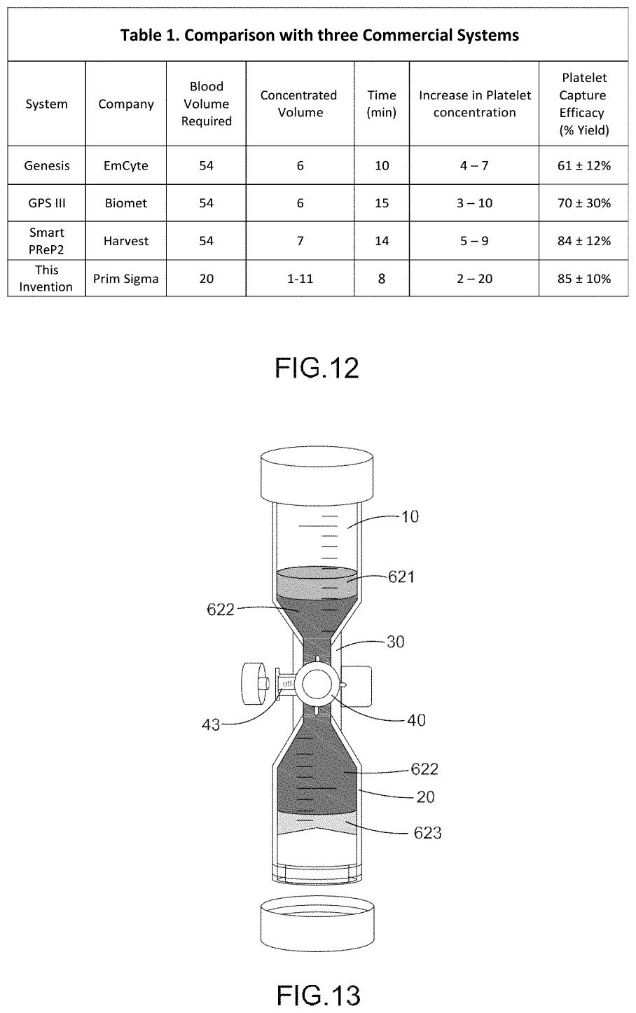

FIG. 12 (Table 1) shows the comparison with some commercial systems in terms of volume requirement, time spent, concentration capability, capture efficiency, and etc. The data for comparison was from Le A D K, Enweze L, DeBaun M R, and Dragoo J L. Current Clinical Recommendations for Use of Platelet-Rich Plasma. Curr Rev Musculoskelet Med. 2018 December; 11(4): 624-634. The data from the invention was collected from ten volunteers. After the PRP was harvested, the data were determined using the same methods as Degen R M, Bernard J A, Oliver K S, Dines J S. Commercial separation systems designed for preparation of platelet-rich plasma yield differences in cellular composition. HSS J. 2017;13:75-80. It is clearly demonstrated that the present invention requires a smaller volume of blood, takes only ten minutes to prepare, and produces consistent results with high capture efficiency. Moreover, the capability of concentrating PRP is easily adjustable, since the PPP could be simply removed from the first container 10 according to specific requirements. To significantly concentrate the platelets in the PRP, the PPP could be suctioned out from the top of the upper first container 10 with minimal disturbance. Because the stopcock valve 40 effectively closes the contaminating RBC off at the bottom, as shown in FIG. 10, more than 90% of PPP could be removed and discarded without disrupting the “buffy coat”. Removal of PPP could be done with a long needle or cannula coupled to a syringe. This way it provides anywhere from 2 to 20 times concentration for the platelets in the final PRP.

Additionally, it is easy to harvest LR-PRP or LP-PRP by manipulating the positions of the “buffy coat” at the threshold of the lower valve port of the three-way stopcock valve 40, as described earlier.

### SAMPLE PREPARATION EXAMPLE 2

As another example of the application of the system of the present invention, FIGS. 13,14 and 15 illustrate a procedure for fat graft preparation from lipoaspirates by the system for liquid component fractionation according to the above preferred embodiment of the present invention.

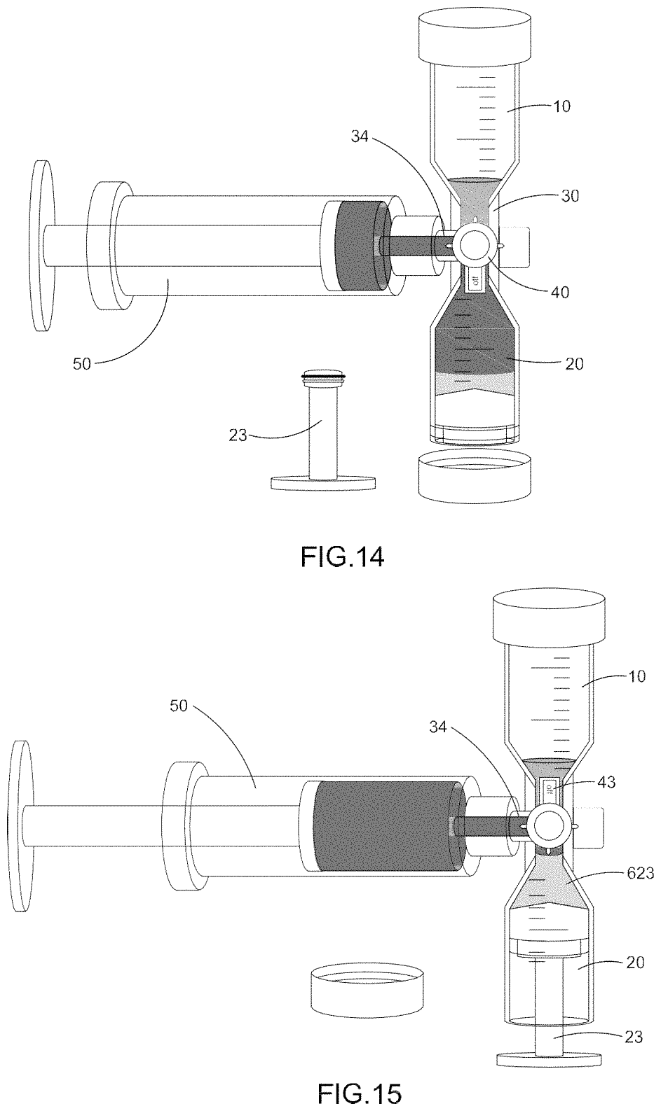

FIG. 13 shows the system of the present invention containing fat after being centrifuged. The centrifugation speed could be anywhere from 100 g to above 3000 g, preferably to use 500 g for 5 to 10 minutes. After the collection syringe 50 is connected to the collection outlet member 34 and the valve lever 43 is turned to block the passage to the second container 20, the fat content is withdrawn from the first container 10. Because the fractionated layers including oil 621, fat 622 and infranatant 623 are quite stable, most of the fat could be removed without disturbing the oil layer 621 above, as shown in FIG. 14. After most of the fat is collected from the first container 10, the valve lever 43 is turned up to block the passage to the first container 10 and the collection syringe 50 is then used to withdraw fat from the second container 20, leaving behind all the water and the RBC at the bottom of the tube and avoiding contamination, as shown in FIG. 15. All in all, this invention provides a simple and clean approach for completely separating fat from oil and water.

### SAMPLE PREPARATION EXAMPLE 3

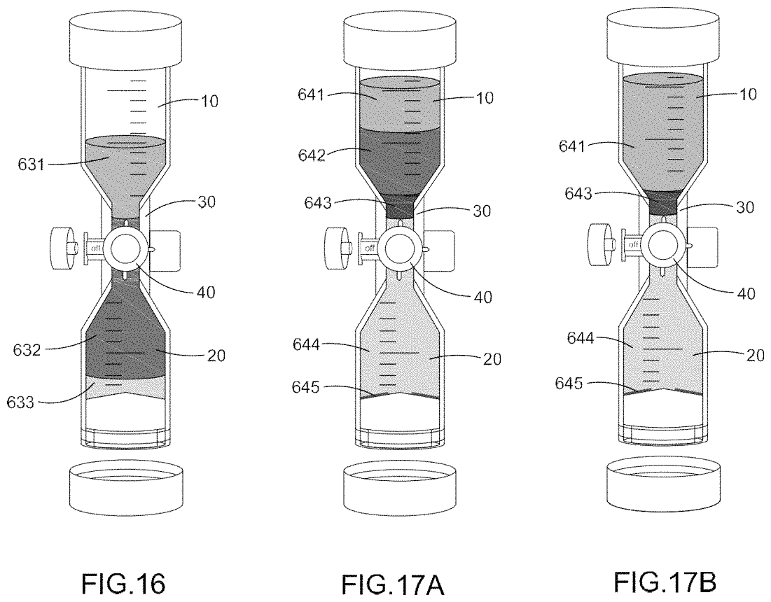

FIG. 16 is a schematic view illustrating a procedure for nano fat processing by the system for liquid component fractionation according to the above preferred embodiment of the present invention. Nano fat is created by first shuffling the harvested fat 30 times through a small “holed” (1.2 mm) Tulip LuerTransfer® (Tulip Medical Products, San Diego, Calif.) and then, subjecting the resulting fat to be pressed through a 400 micron screen. The recommendation is to use the emulsified product directly. However, this practice may not be sound considering what the components are after a centrifugation process: in addition to the infranatant, the amount of oil on the top is much more (FIG. 16) than the preparation of regular fat samples. Almost 30 to 50% of the fat will be destroyed by the shuffling and screening processes, releasing free lipids. It is found that this oil is even harder than the oil in FIG. 2 to remove, probably due to the fact that emulsification took place before the centrifugation. In any case, this oil byproduct should ideally be isolated out before a quality fat grafting is performed.

The fractionation starts with a centrifugation with the same setting as in the above second example and FIG. 16 shows the resulting product. By using a syringe to draw out the nano fat the same way as that in FIGS. 13 and 14, the nano fat could easily be isolated. The precision of the valve cutting off the oil contamination makes this invention a very valuable addition to precision fat grafting medicine.

### SAMPLE PREPARATION EXAMPLE 4

FIGS. 17A and 17B are perspective views illustrating a procedure for Autologous fat stromal vascular fraction (SVF) harvesting with the system for liquid component fractionation according to the above preferred embodiment of the present invention. Autologous fat stromal vascular fraction (SVF) contains most of the stromal stem cells. It could be prepared mechanically by ultrasonic sonication, homogenization, fine trituration, mortar and pestle treatment, etc. Because most of these treatments require addition of a buffer at some point, the water content is typically higher than those see in the above second and third examples. Specifically, after the fat is processed, about an equal amount of a buffer such as PBS is added. After centrifugation, the components in the tube system of this embodiment could look like those in FIG. 17A or FIG. 17B. The fractionated layers include oil 641, fat 642, a pale-colored layer 643, infranatant 644, and a SVF layer 645, with much more content as water, typically occupying the whole second container 20. The differences in the “fat” contents between FIGS. 17A and 17B lie in the fact that the two samples are sonicated to different levels, with the sonication of fat in FIG. 17A being gentler, and FIG. 17B being more complete.

Traditionally, it has always been a difficult task to harvest SVF in a clean way. While the numbers of SVF cells are in the ranks of 10⁵ to 10⁶ per ml of fat, the single cell numbers in the pale-colored layer and the fine nano fat layers are in the ranks of 10⁹. Even a diminutive, almost ignorable amount of contamination of the SVF by the above-mentioned fractions would drastically undermine the validity of the number of the SVF cells counted at the end, and therefore, the subsequent treatment regimens or related experiments. This kind of contamination takes place very easily and very frequently with most, if not all, of the mechanical systems currently in use, for reasons such as the contaminating fractions sticking to the walls of the tube/bottle, or some almost invisible small pieces breaking away from the main fraction layer during the SVF collection. These are further complicated by the fact that the morphology of pre-adipocytes and small fat cells are not much different from the SVF cells when in suspension. Considering these, it is no wonder that the numbers of harvested “stem cell” from fat vary from 10⁴ to 2.24×10⁷ among different publications. And this fact significantly hampers the progress in the field of fat stem cell research and clinical application. As far as we know, currently, there is no method or commercially available mechanical system for the purpose of purifying SVF without any risk of cell contamination by the other fractions.

With the present invention, by manipulating the valve positioning, it is easy to flush out the pelleted SVF. The only requirement is to pre-calculate the amount of water that might exist in the final preparation, so as to let the water occupy the whole second container 20 and some on the lower portion of the first container 10.

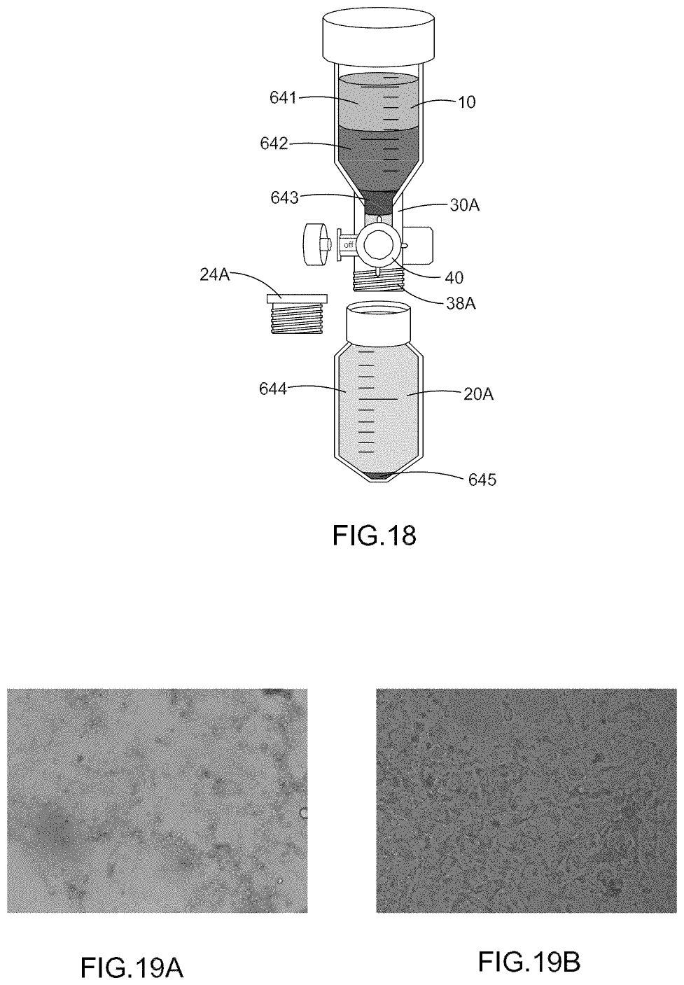

Theoretically, there are much more SVF cells in the fat than the small number of cells existing in the pellet, as shown in FIGS. 17A, 17B and 18. However, most of the SVF cells are stuck in the fat/vascular aggregates in the “pale-colored layer”. Unless a much more effective mechanical method is invented, these SVF cells remain attached to the aggregates. Because the “Pale”-layer is rich in stem cells, this particular fraction, could be separately harvested after SVF is collected. The “Pale” layer could then be used to treat various medical conditions.

In any case, since the SVF need to be flushed out and re-pelleted for harvesting, it might be more convenient to use a first alternative mode of the above preferred embodiment, as shown in FIG. 18, which is slightly different in that the second container 20A is now designed into a detachable upright-positioned conical tube, with the top part having internal screw threads. When this conical tube container is detached, its top could be sealed with a second cap 24A. During the shipping, storage and fat processing, however, the bottom conical tube is twisted tightly onto a connecting end portion 38A of the tunnel connecting tube 30A below the three-way stopcock valve 40. As can be seen, the SVF pellet can now be easily harvested after the infranatant is either decanted or suctioned out. This latter design is particularly useful for liquids that contain desirable components that exist in a pellet form or in the lower part of the second container 20.

Phase-contrast microscopic photos in FIGS. 19A and 19B show the cell culture of the first generation of the fat SVF harvested using this modified system. It is interesting to notice that while many cells undergo proliferation, some cells just stayed there and never propagated, consistent with the cell composition in the SVF preparation.

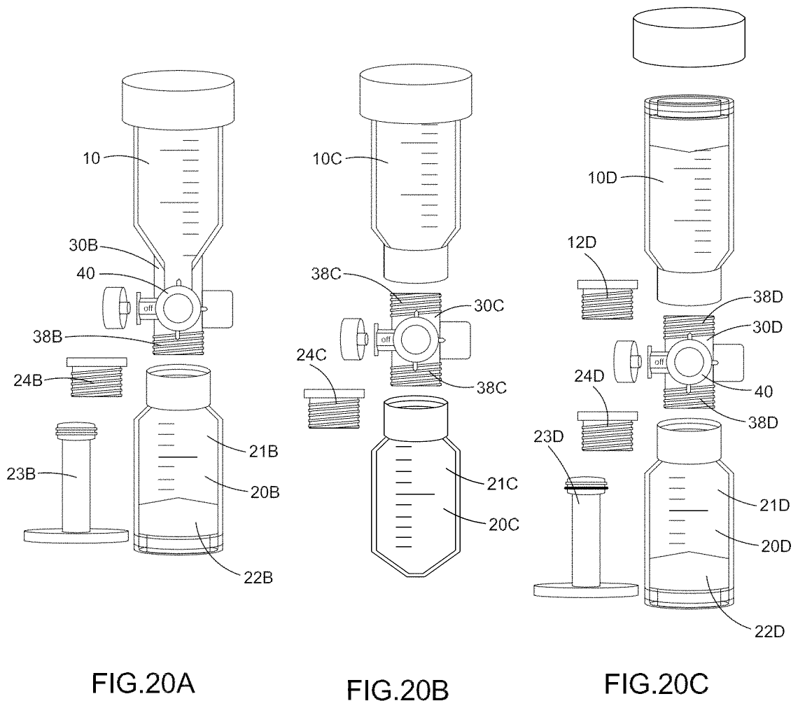

FIGS. 20A, 20B and 20C are perspective views illustrating the system for liquid component fraction according to a second, a third and a fourth alternative modes of the above preferred embodiment of the present invention, wherein the figures show various modification based on the previously mentioned tube designs, turning fixed connections with the 3-way stopcock valve to completely detachable ones. The core of the designs is that the 3-way stopcock 40 is turned into a double male connector on both ends, using screw threads to connect with a conical tube structure or a syringe type of tube piece on one end, and a similar or different piece on the other. The interchangeability based on a double-male-connector of the three stopcock valves makes designing new systems for sample fractionation tasks much more versatile and convenient.

More specifically, as shown in FIG. 20A of the drawings, the system comprises a first container 10, a second container 20B, a tunnel connecting member 30B, and a stopcock valve 40. The second container 20B comprises a second container body 21B which is embodied as a bottle, a movable member 22B, a plug member 23B, and a second cap 24B which is adapted for being secured on top of the second container body 21B. The tunnel connecting member 30B comprises a connecting end portion 38B below the three-way stopcock valve 40, and this end portion 38B is embodied as a threaded connecting portion for engaging with the top of the second container body 21B.

As shown in FIG. 20B of the drawings, the system comprises a first container 10C, a second container 20C, a tunnel connecting member 30C, and a stopcock valve 40. The second container 20C comprises a second container body 21C which is embodied as a conical tube, and a second cap 24C which is adapted for being secured to cover the top of the second container body 21C. The tunnel connecting member 30C comprises two connecting end portions 38C at both ends of the tunnel connecting tube 30C, for engagement with the bottom of the first container body 11C and the top of the second container body 21C.

As shown in FIG. 20C of the drawings, the system comprises a first container 10D comprising a first cap 12D, a second container 20D, a tunnel connecting member 30D, and a stopcock valve 40. The second container 20D comprises a second container body 21D which is embodied as a bottle, a movable member 22D, a plug member 23D, and a second cap 24D. The tunnel connecting member 30D comprises two connecting end portions 38C at both ends of the tunnel connecting tube 30D, for engagement with the bottom of the first container body 11D and the top of the second container body 21D.

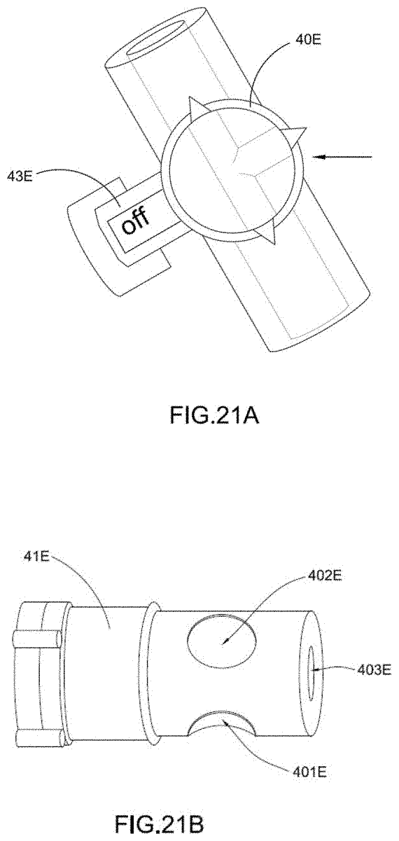

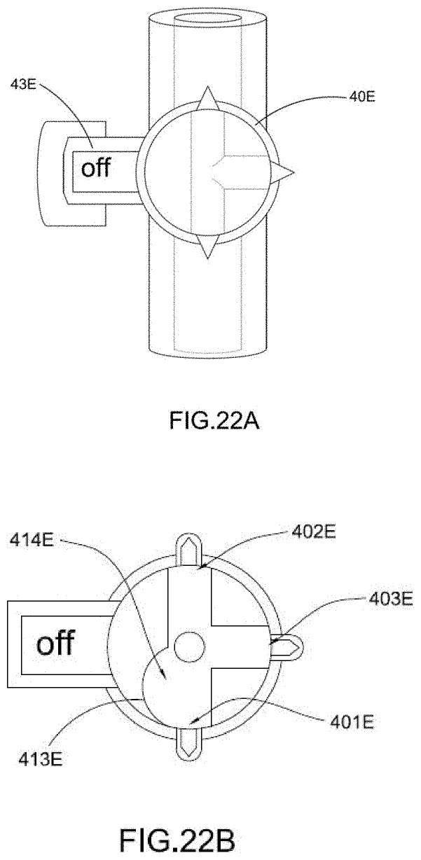

FIGS. 21A to 22B are schematic views illustrating a stopcock valve of the system for liquid component fractionation, modification could be made for one of the valve ports on a valve plug 41E of a three-way stopcock valve 40E, according to a fifth alternative mode of the above preferred embodiment of the present invention.

Specifically, the port 401E facing the second container 20, when the valve lever 43E is aligned with the collection outlet member 34, could be modified. FIG. 21B shows the side view of the valve plug 41E turned 90 degrees clockwise from the frontal view of the three-way stopcock vale as in FIG. 2 and FIG. 21A. Now viewing at the valve plug 41E from the side, following the viewing direction of the arrow shown in FIG. 21A, one can see the specified valve port at the bottom of the valve plug 41E. This valve port 401E could be made differently from the other two valve ports 402E and 403E such that the far end edge could be made beveled-in inside, forming a round-out space inside the valve passageway, as shown in FIG. 22B. In other words, the valve plug 41E comprises a scoop portion 413E for defining a round-out space 414E inside the valve plug 41E.

FIG. 22A shows the transparent frontal view of the tunnel connecting member 30 and the stopcock valve 40E. FIG. 22B is a magnified, transparent view of the three-way stopcock valve 40E from the same frontal view. Note that this specified port 401E is different from valves 402E and 403E in that there is a created round-out space 414E. However, the size and shape of the port opening is not altered. This extra beveled-in space helps to facilitate scooping up the desired “buffy coat” during PRP isolation. When the “buffy coat” is pushed up through the boarder of the valve port 401E, the easier flow dynamics, rendered by the beveled “scoop” inside and behind the edge of the valve port 401E, enhances the ability of the system to collect more of the “buffy coat” yet minimizing the RBC contamination from below. This specially designed valve feature is not limited to being used for PRP purification. Any other fractionation of substances that presents the target fraction to this particular port could benefit significantly from the better dynamics of this unique design.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A system for liquid component fractionation, comprising:
a first container having a first containing cavity;
a second container having a second containing cavity;
a tunnel connecting member, provided between said first container and said second container, comprising at least one tunnel body having one or more connecting tunnels communicating said first containing cavity with said second containing cavity, and a collection outlet member having a collection port coupled to said at least one tunnel body; and
a stopcock valve, provided at said tunnel connecting member, having three ports configured to selectively align with said one or more connecting tunnels and said collection port.

2. The system, as recited in claim 1, wherein said connecting tunnel has a first passage communicated to said first containing cavity and a second passage communicated to said second containing cavity, wherein said stopcock valve is rotatably disposed at said tunnel connecting member to allow only two of said first passage, said second passage and said collection port to align with two of said three ports respectively.

3. The system, as recited in claim 1, wherein said connecting tunnel has a first passage communicating with said first containing cavity and a second passage communicating with said second containing cavity, wherein said stopcock valve is rotatably disposed at said tunnel connecting member to move between a position selected from the group consisting of a first position allowing said first passage and said second passage to communicated with two of said three ports, a second position allowing said first passage and said collection port to communicate with two of said three ports, and a third position allowing said second passage and said collection port to communicate with two of said three ports.

4. The system, as recited in claim 1, wherein said stopcock valve is rotatably disposed at said tunnel connecting member to move between a first position allowing one of said three ports to align with said collection port and a second position blocking said three ports from aligning with said collecting port.

5. The system, as recited in claim 4, wherein said second container comprises a second container body defining said second containing cavity, a movable member disposed in said second container body, and a plug member that is operable to engage with said movable member to push said movable member.

6. The system, as recited in claim 5, wherein said a movable member comprises a movable plunger tip and an engaging member supporting said movable plunger tip, wherein said plug member is operable to engage with engaging member to push said movable member.

7. The system, as recited in claim 2, wherein said second container is a conical tube.

8. The system, as recited in claim 2, wherein said first container, said second container, said tunnel connecting member, and said stopcock valve are formed as an integral one-piece structure.

9. The system, as recited in claim 3, wherein at least one of the said first container and said second container is detachably mounted with said tunnel connecting member.

10. The system, as recited in claim 3, wherein said stopcock valve has a diameter ranged 3 mm to 15 mm.

11. The system, as recited in claim 4, wherein said stopcock valve comprises a valve plug defining said three ports, and a valve lever that is operated for rotating said stopcock valve, three valve port identifiers for identifying said three ports, and an “off” position identifier for identifying said valve lever.

12. The system, as recited in claim 3, wherein said stopcock valve comprises a valve plug having said three ports, wherein said valve plug could be modified to comprise a scoop portion defined as a round-out space inside said valve plug that is capable of being moved to a position aligned with said second passage to communicate said round-out space with said second container.

13. The system, as recited in claim 2, wherein each of said first and second container is a tube having a slope surface transitioning from each of said first and second containers to said tunnel connecting member, requires a transition angle ranging from 100 to 170 degrees.

14. The system, as recited in claim 13, wherein said transition angle ranges from 120 to 150 degrees.

15. The system, as recited in claim 4, wherein said stopcock valve comprises a valve plug defining said three ports, a valve lever that is operated for rotating said stopcock valve, and a blocking member which comprises a static blocking element located on said tunnel connecting member, and a mobile blocking element that is located behind said valve lever on said valve plug for moving along with said valve plug, wherein said static blocking element and said mobile blocking element working to block each other to allow said valve lever to only move 180 degrees, in order to prevent a communication of said collection port with both said first container and said second container at the same time.

16. The system, as recited in claim 1, wherein said tunnel connecting member comprises only one said tunnel body defining said connecting tunnel and a side wing at a side of said tunnel body.

17. A method for liquid component fraction through a system which comprises a first container, a second container, a tunnel connecting member provided between said first container and said second container, and a stopcock valve provided at said tunnel connecting member, wherein the method comprises the steps of:
(a) centrifuging said system which is filled with a liquid to separate said liquid into a plurality of fractionation layers; and
(b) operating said stopcock valve to allow one of said plurality of fractionation layers to be collected through a collection outlet member of said tunnel connecting member.

18. The method, as recited in claim 17, wherein the method is arranged for isolating plate-rich plasma (PRP) from blood, wherein in step (a), the blood is separated into three fractionation layers comprising a platelet-poor plasma layer, a buffy coat layer in a middle containing the plate-rich plasma, and a layer of red blood cells, wherein in step (b), said stopcock valve is switched to block the said layer of red blood cells in said second container, and said layer of buffy coat is collected through said collection outlet member.

19. The method, as recited in claim 17, wherein the buffy coat layer comprises a leukocyte-rick platelet-rich plasma layer and a leukocyte-poor platelet-rich plasma layer, wherein the method further comprises a step of manipulating positions of the buffy coat layer to separate the leukocyte-poor platelet-rich plasma layer with the leukocyte-rich platelet-rich plasma layer.

20. The method, as recited in claim 19, wherein the step (b) further comprises a step of aligning the scoop portion with a round-out space in said valve plug of said stopcock valve with said second container to communicate said round-out space with said second container for harvesting the leukocyte-poor platelet-rich plasma layer.

21. The method, as recited in claim 17, wherein the method is arranged for fractionate lipoaspirates, wherein in the step (b), said stopcock valve is switched to facilitate collection of fat through said collection outlet member.

22. The method, as recited in claim 21, further comprising a step of mechanically shuffling said liquid before the step (a), wherein in step (b), said stopcock valve is switched to facilitate collection of nano fat through said collection outlet member.

23. The method, as recited in claim 17, wherein the method is arranged for harvesting autologous fat stromal vascular fraction, wherein in the step (a), said liquid is separated into fractionation layers comprising an oil layer, a fat layer, a pale-colored layer, a layer of infranatant, and a fat stromal vascular fraction pallet, wherein the fat stromal vascular fraction pallet is collected at a bottom of said second container.

24. The method, as recited in claim 23, wherein the step (b) further comprises the steps of:
(b-1) manipulating down a level of the pale-colored layer until an interface between said fat layer and said pale-colored layer reaches an upper edge of a port positioned at a top of said stopcock valve,
(b-2) switching a valve lever of said stopcock valve to block flow from said first container, and
(b-3) collecting said pale-colored layer through said collection outlet member.

25. The method, as recited in claim 23, wherein the step (b) further comprises the steps of:
(b1) switching a valve lever of said stopcock valve to close off from said second container, and
(b2) detaching said second container from said tunnel connecting member to harvest said infranatant layer and said fat stromal vascular fraction layer, when said system has a detachable second container.

26. The method, as recited in claim 23, wherein the step (b) further comprises the steps of:
(b.1) manipulating down a level of said pale-colored layer until an interface between said fat layer and said pale-colored layer reaches a lower edge of said port positioned at a bottom of said stopcock valve,
(b.2) switching a valve lever of said stopcock valve to block flow from said second container, and
(b.3) collecting said pale-colored layer through said collection outlet member.

27. The method, as recited in claim 17, wherein said liquid has difference in relative weights among the plurality of fractionation layers.
